(19) United States
(12) Patent Application Publication
(10) Pub. No.: US 2019/0192854 A1
(43) Pub. Date: Jun. 27, 2019

(54) System for Artificial Retina Prosthesis

(71) Applicant: IRIDIUM MEDICAL TECHNOLOGY CO., LTD., Hsinchu (TW)

(72) Inventors: Feng-Hsiung HSU, Hsinchu (TW); Yung-Chan CHEN, Hsinchu (TW); Long-Sheng FAN, Hsinchu (TW)

(21) Appl. No.: 16/230,218

(22) Filed: Dec. 21, 2018

Related U.S. Application Data
(60) Provisional application No. 62/610,004, filed on Dec. 22, 2017.

(51) Int. Cl. A61N 1/36; H04N 5/378; A61N 1/05; A61N 1/02

(52) U.S. Cl. CPC A61N 1/36046; A61N 1/025; A61N 1/0543; H04N 5/378

(57) ABSTRACT

The present invention relates to a system for artificial retinal prosthesis comprising a pixel array, a correlated double sampling unit, an analog-to-digital converter, a digital core, and a digital-to-analog converter. The system stimulates retinal cells row-to-row, and therefore can effectively reduce large transient currents and avoid unfavorable condition of power drop due to large transient currents.

### CROSS-REFERENCE TO RELATED APPLICATION

The present application claims the benefit of U.S. Provisional Patent Application No. 62/610,004, entitled “System for Artificial Retina Prosthesis,” which was filed on Dec. 22, 2017, and the disclosure of which is incorporated herein by reference in its entirety.

### FIELD OF THE INVENTION

The present invention relates to a medical device, and more particularly to an implantable medical device capable of stimulating nerve cells.

### BACKGROUND OF THE INVENTION

Currently, among the patients with visual deterioration, some patients choose to implant an artificial retina to improve their vision. At present, expensive artificial retinas of the commercial standard with low pixels have a limited improvement on the quality of life of patients. In view of this, many companies and research institutes have begun to actively invest in the improvement of microsystem for artificial retina.

However, the conventional artificial retinal devices are mostly microelectrodes made of planar chips, which are mismatched with the non-planar shape of the retinal tissues, and may cause additional interference between the microelectrodes, and adversely affect the image resolution of the components. In this regard, the applicant's U.S. Pat. No. 9,155,881 B2 proposes a non-planar chip set having a flexible structure formed by a curved deformation of a planar shape. The flexible structure comprises at least one semiconductor material layer, around a central portion of the flexible structure, there is a plurality of slit passage openings extending from a periphery of the flexible structure toward the central portion, and the slit passages are used to reduce a displacement stress generated after the planar shape is crookedly deformed to become the flexible structure. Outside the flexible structure, a bonding structure is combined with at least one fixing structure to maintain the flexible structure in a curved state, and the element can be thin enough to be bent 90 micrometers from a center to an edge to match the shape of the retina. In this way, a neuron-to-electrode distance between the component electrodes and target nerve cells of the retina is reduced, and the electric power required for activating or stimulating each pixel of the nerve cells can be reduced to generate a higher pixel density with a supplyable power density, and can also improve the image resolution received by the nerve cells of the user that are implanted with the components.

However, the improvement on the artificial retina is not limited to this. In order to give the user a more comfortable visual experience, many R&D teams are actively making improvements on the image resolution. The current mainstream method is to increase a number of the pixel electrodes of artificial retina, but the complicated circuit and signal processing that come with it become a new problem.

For example, in an artificial retina having a plurality of pixel units, disclosed in U.S. Pat. No. 7,751,896 B2, each of the pixel units comprises at least one image unit for converting an incident light into an electrical signal, and at least one amplifier, wherein the image unit has a logarithmic characteristic that converts an incident light of a specific intensity into an electrical signal of a specific amplitude. Therefore, the incident light can be efficiently converted into a stimulation signal by a simple circuit device, and the nerve cells in the retina can be effectively stimulated even if given different ambient illuminations.

For example, in an artificial retina disclosed in U.S. Pat. No. 6,804,560 B2, at least one amplifier is provided in the artificial retina, and a plurality of stimulation electrodes is provided via the at least one amplifier based on signals received by a pixel element. The patented artificial retina further comprises at least one photosensitive reference element coupled to the amplifier, the photosensitive reference element is capable of controlling a magnification of the amplifier based on an amount of light energy irradiating thereon. In this way, electrical stimulation signals of discharge are suitable for average light intensity, just like the response of eye to ambient light conditions under natural conditions, not only avoiding the stimulation electrodes from transmitting too strong electrical signals to adjacent retinal nerve cells under relatively bright ambient light, resulting in excessive stimulation or even cell damage; on the other hand, stimulation signals with sufficient intensity can be transmitted to adjacent retinal nerve cells even under very weak ambient light conditions.

In view of a number of pixel units of the artificial retina continues to increase, investing continuously in related research on improvements of suchlike circuits and signal processing is urgently required.

### SUMMARY OF THE INVENTION

A main object of the present invention is to solve the complicated circuit and signal processing problems associated with the addition of pixel electrodes of artificial retina.

In order to achieve the above object, the present invention provides a system for artificial retinal prosthesis comprising a pixel array, the pixel array comprises n sub-pixels for converting incident light to electrical stimulation signals; a correlated double sampling unit, the correlated double sampling unit has a communication connection with the pixel array electrically; an analog-to-digital converter, the analog-to-digital converter is coupled to the correlated double sampling unit and outputs a first digital signal; a digital core, the digital core is coupled to the analog-to-digital converter to receive the first digital signal and output a second digital signal after analysis; and a digital-to-analog converter, the digital-to-analog converter is coupled to the digital core to receive the second digital signal.

The present invention further provides a system for artificial retinal prosthesis, comprising a retinal implant device. The retinal implant device comprising a pixel array and a control circuit for controlling the pixel array to output at least one electrical stimulus to a retinal nerve cell, wherein the control circuit stimulates the retinal nerve cell row-to-row to reduce large transient currents.

The conventional techniques simultaneously output current to all of the pixel electrodes. In the case where a number of the pixel electrodes included in the artificial retina is small, the above method of simultaneously outputting current does not cause too much problem; but in recent years, a number of the pixel electrodes in artificial retina has gradually increased to several thousands, and in the case of simultaneously outputting current, the problem of excessive transient currents will occur. Therefore, in comparison with the conventional techniques being incapable of stimulating retinal nerve cells row-to-row, the present invention can effectively reduce large transient currents and avoid unfavorable condition of power drop due to large transient currents by stimulating retinal nerve cells row-to-row. In addition, by having the digital core disposing in the system for artificial retinal prosthesis of the present invention, a large amount of data can be quickly processed and analyzed, which is especially suitable for the weight calculation of electrical stimulation signals.

### BRIEF DESCRIPTION OF THE DRAWINGS

FIG. 1 is a schematic view of an architecture of a system for artificial retinal prosthesis of the present invention;

FIG. 2 is a schematic view of a circuit architecture in a sub-pixel according to an embodiment of the present invention;

FIG. 3 is a plan view of the system for artificial retinal prosthesis according to an embodiment of the present invention; and

FIG. 4 is a plan view of the system for artificial retinal prosthesis according to another embodiment of the present invention.

### DETAILED DESCRIPTION OF THE PREFERRED EMBODIMENTS

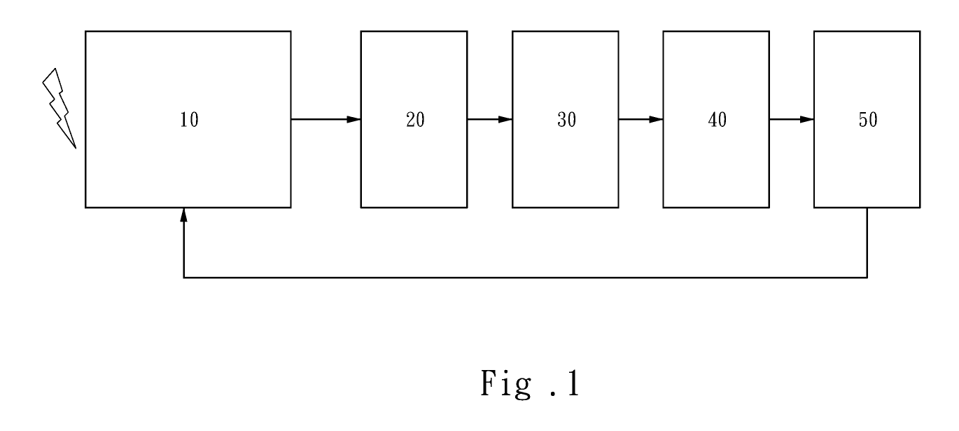

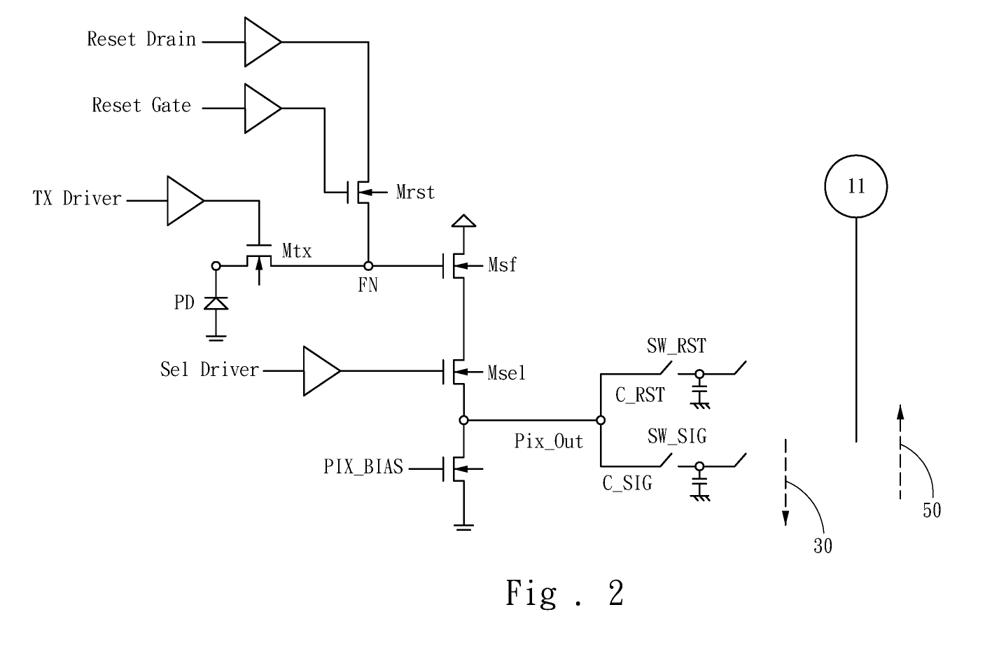

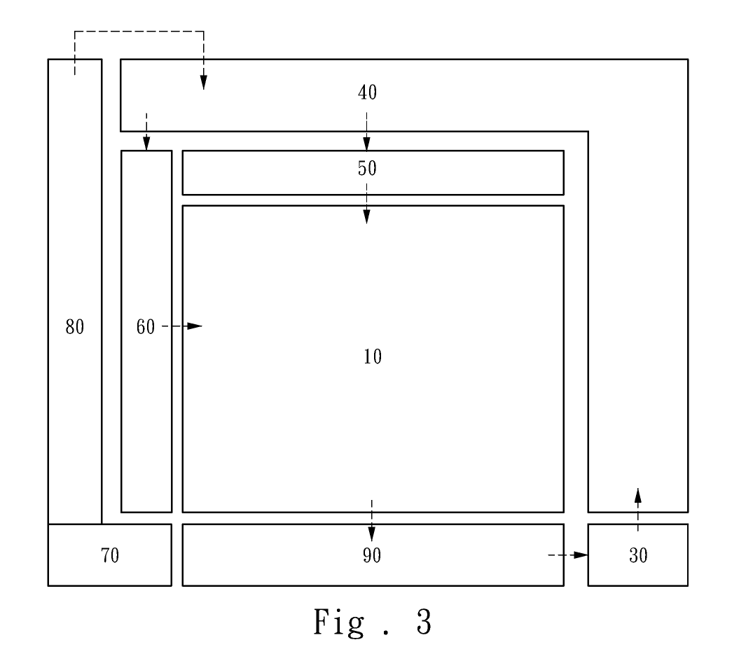

The detailed description and technical contents of the present invention will be described as follows in conjunction with FIG. 1, FIG. 2 and FIG. 3.

FIG. 1 is a schematic view of an architecture of a system for artificial retinal prosthesis of the present invention, which mainly comprises a pixel array 10, a correlated double sampling (CDS) unit 20, an analog-to-digital converter (ADC) 30, a digital core 40, and a digital-to-analog converter (DAC) 50. In this embodiment, the pixel array 10, the correlated double sampling unit 20, the analog-to-digital converter 30, the digital core 40, and the digital-to-analog converter 50 are integrated on a single silicon substrate to form a chip, which can be disposed in a sub-retina portion or an epi-retina portion of an eye structure, and the present invention is not particularly limited thereto.

The pixel array 10 comprises a plurality of sub-pixels, and the number of sub-pixels is n. Each of the sub-pixels comprises a pixel electrode 11, a photosensitive region including a photodiode (PD), and a circuit architecture electrically connected to the photodiode. The pixel electrode 11 is connected to the digital-to-analog converter 50 and can stimulate at least one retinal nerve cell, and analog signals sent from the digital-to-analog converter 50 are transmitted to the at least one retinal nerve cell for electrical stimulation. The above n can be an integer between 500 and 50,000, and n in this embodiment is between 3,500 and 5,000, for example, about 4,000.

Referring to FIG. 2, FIG. 2 shows a circuit architecture in which the pixel electrode 11 and the correlated double sampling unit 20 are integrated in an embodiment of the present invention. In operation, the circuit architecture performs reset, exposure and read out of the pixel electrode 11, and sampling of the correlated double sampling unit 20. Detailed description is as follow.

Reset

When one of the sub-pixels starts to operate, SW_RST and SW_SIG are both disconnected, so a signal of Pix_Out is not stored on capacitors C_RST and C_SIG. Before exposure, Reset Drain, Reset Gate and TX Driver in the circuit architecture are first turned on. At this time, a voltage of Reset Drain will be written into the photodiode through Mrst and Mtx. This step is mainly to clear electrons in the photodiode to allow the photodiode to start exposure. In addition, since current Sel Driver is in the off state, it represents Msel is also turned off, so Pix_Out does not have any signal, and since SW_RST and SW_SIG are both disconnected at this time, Pix_Out without any signal will not have any effect.

Exposure

When reset is complete, TX Driver will be turned off. At this time, the photodiode becomes a floating node, and can start to store electrons. When the exposed light (i.e. the received light) is stronger, the more electrons are stored, and the value a voltage FN is lower.

Sampling

Firstly, Mtx is turned off, Mrst is turned on, Msel is turned on, SW_RST is connected, and SW_SIG is disconnected. As a result, Pix_Out_rst (representing Pix_out when Mrst is turned on) will satisfy the following formula:

Pix_Out_rst=FN_RST−VGS_Msf−VDS_Msel (Formula 1)

Wherein, FN_RST represents the FN voltage at reset, VGS_Msf represents a gate-to-source voltage of Msf, and VDS_Msel represents a drain-to-source voltage of Msel. At this time, a voltage of Pix_Out_rst will be stored in the capacitor C_RST.

Then, Mtx is turned on, Mrst is turned off, Msel is turned on, SW_RST is disconnected, and SW_SIG is connected. As a result, Pix_Out_sig (representing Pix_out when PD receives light exposure) will satisfy the following formula:

Pix_Out_sig=FN_SIG−VGS_Msf−VDS_Msel (Formula 2)

Wherein, FN_SIG represents the information of a voltage relative to a light intensity stored in the photodiode, and Pix_Out_sig will be stored in the capacitor C_SIG.

Signals of the capacitors C_RST and C_SIG are sent to a pre-stage circuit of the analog-to-digital converter 30, and a difference between the two signals are extracted. The difference between the two signals is

Pix_Out_rst−Pix_Out_sig=FN_RST−FN_SIG (Formula 3)

It can be seen that the effects of VGS_Msf and VDS_Msel are removed, leaving only FN_RST and FN_SIG, thereby deducting the associated noise and reducing the mismatch. The correlated double sampling unit 20 acts as a noise reduction element for removing unwanted offsets in the signals. In this embodiment, the noise in the light-induced electrical stimulation signal is removed by the correlated double sampling unit 20.

Read Out

In this way, when the photodiode receives incident light, the photodiode convert the incident light to a plurality of light-induced electrical stimulation signal through the photodiode according to an intensity ratio of the incident light, and the light-induced electrical stimulation signal is outputted to the analog-to-digital converter 30 via the node Pix_Out.

The light-induced electrical stimulation signal processed by the correlated double sampling unit 20 is transmitted to the analog-to-digital converter 30, and is converted into a first digital signal. Then, the light-induced electrical stimulation signal is outputted. The analog-to-digital converter 30 suitable for use in the present invention is not particularly limited. For example, the analog-to-digital converter 30 may be a pipeline ADC or a column-parallel ADC.

After the first digital signal is received by the digital core 40, an analysis process is performed to define an appropriate gain and offset for the subsequent digital-to-analog converter (DAC) 50, and a second digital signal is outputted.

The digital-to-analog converter (DAC) 50 receives the second digital signal and converts it to an appropriate analog signal according to the second digital signal, and transmits the analog signal back to the pixel array 10 to stimulate the at least one retinal nerve cell.

Referring to FIG. 3, in an embodiment, the system for artificial retinal prosthesis further comprises a decoder 60, a wireless unit 70, a power and bandgap unit 80, and a column decoder and pixel biasing unit 90. Wherein the decoder 60 can be a row decoder 60, as shown in FIG. 3; or include a first decoder and a second decoder. When the row decoder 60 is employed, the row decoder 60 can respectively output a photosensitive switching signal and a stimulation switching signal to the pixel array 10 to control the pixels of each row to be turned on or off at an appropriate time for photoreception and/or stimulation. When the first decoder and the second decoder are employed, the former can be used to output the photosensitive switching signal, and the latter is used to output the stimulation switching signal.

The wireless unit 70 is used to receive an external wireless signal, such as a wireless alternate current signal, and the wireless alternate current signal can include a power signal and/or a command signal. For example, if the power signal and the command signal are included in the wireless alternate current signal, the wireless unit 70 converts the power signal in the wireless alternate current signal into a DC voltage, and then transmits the DC voltage to the power and bandgap unit 80. The power and bandgap unit 80 converts the DC voltage into a stable voltage to provide power required for operation of the system. And the wireless unit 70 extracts the command signal in the wireless alternate current signal and transmits the command signal to the digital core 40. In this embodiment, the column decoder and the pixel biasing unit 90 are integrated into one component, but in other embodiments, the component can also be split into the column decoder and the pixel biasing unit independently of each other.

Regarding the analog-to-digital converter 30, if a pipeline analog-to-digital converter is used, the analog-to-digital converter 30 converts only a certain pixel of a row each time the conversion is performed, and after each pixel of the row is converted, the digital core 40 controls the row decoder 60 to select the pixel array 10 to jump to a next row, and the information of the row is transmitted to the column decoder and pixel biasing unit 90. The column decoder and pixel biasing unit 90 then sequentially transmits each pixel of the row to the analog-to-digital converter 30 one by one for analog-to-digital conversion. After the image information of an entire picture is converted and stored in the digital core 40, the digital core 40 can have the image information of the entire picture, and corresponding stimulation parameters (the second digital signals) are generated after analysis, and start to stimulate the at least one retinal nerve cell row-to-row through the digital-to-analog converter 50 and the row decoder 60. The digital-to-analog converter 50 is responsible for converting a row of the second digit signals into analog stimulation signals. The row decoder 60 is responsible for selecting which row of the pixel array 10 the analog stimulation signals of this row are to be sent to.

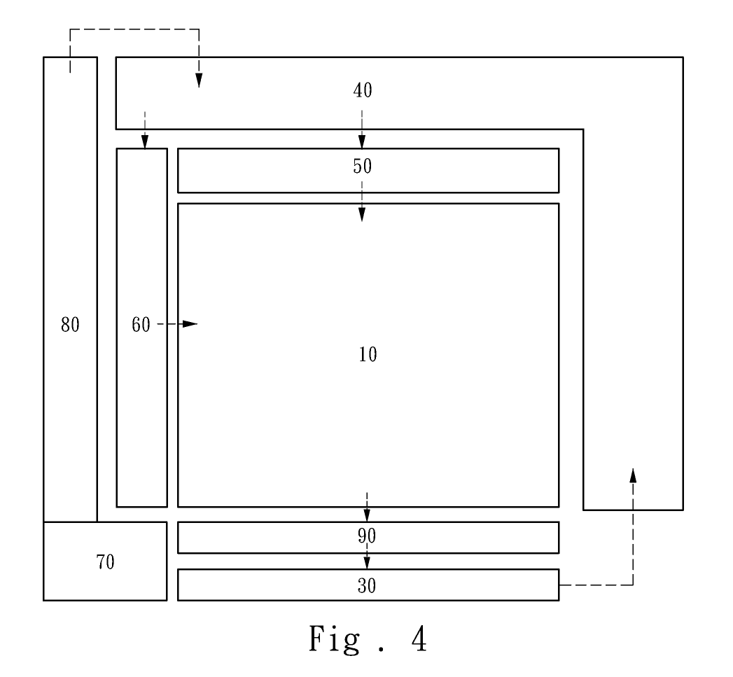

Please refer to FIG. 4, which is a plan view of the system for artificial retinal prosthesis according to another embodiment of the present invention. If a column-parallel analog-to-digital converter is used, the conversion mode of the analog-to-digital converter 30 is to perform analog-to-digital conversion for an entire row at the same time. In other words, in this architecture, signals of pixels of each row are processed in the same time by the pixel biasing unit 90 corresponding to the column and the analog-to-digital converter 30 corresponding to the column. (In this architecture of FIG. 4, the pixel biasing unit 90 doesn't include the column decoder, because pixel signals in a row are processed simultaneously by the analog-to-digital converter 30. The column decoder is only embedded in the analog-to-digital converter 30 because the ADC output of each pixel in a row should be sent to the digital core 40 pixel by pixel sequentially, a column decoder is needed to select which column of pixel in a row should be sent to the digital core 40.) But in FIG. 3, the column decoder is necessary in the unit 90 to select which column of pixel in a row should be sent to the analog-to-digital converter 30.

Returning to the embodiment of FIG. 3, in operation, the digital core 40 first controls the row decoder 60 to output the photosensitive switching signal to the pixel array 10, each row of the pixel array 10 will be sequentially illuminated. According to the photoreception of each column of the pixel array 10, the column decoder and pixel biasing unit 90 outputs a corresponding pixel bias to the analog-to-digital converter 30 for converting the corresponding pixel bias into the first digital signal according to the incident light, and the first digital signal is transmitted to the digital core 40.

After receiving the first digital signal of a whole pixel array, the digital core 40 performs an analysis process, and then generates the second digital signal and transmits the second digital signal to the digital-to-analog converter 50, and then the digital-to-analog converter 50 generates an electrical stimulation signal related to light intensity and sends the electrical stimulation signal to the pixel electrodes 11. At the same time, the digital core 40 also controls the row decoder 60 to output the stimulation switching signal to the pixel array 10 to control the pixels of each row to be turned on or off at an appropriate time, and the electrical stimulation signal is coordinatively used to electrically stimulate the at least one retinal nerve cell.

Specifically, the row decoder 60 is used to control reset, exposure and read out of the pixel array 10, that is, to control Reset Drain, Reset Gate, TX Driver, and Sel Driver in FIG. 2 to reset or expose the pixels of a specific row. When all the pixels are exposed, images of the entire picture can be obtained. After further analysis by the digital core 40 suitable stimulating parameters are generated and sent to the digital-to-analog converter 50 and thus the magnitude of the electrical stimulation signal required for inputting into the pixels of each row to stimulate the at least one retinal nerve cell can be obtained. Secondly, since the present invention stimulates the at least one retinal nerve cell row-to-row, when the digital-to-analog converter 50 of FIG. 3 sends an electrical stimulus, only one row of electrical stimulus is generated within a same time, and at this time, the row decoder 60 must select which row in the pixel array 10 to receive the electrical stimulus.

In another embodiment, the invention provides a system for artificial retinal prosthesis. The system integrates the above-mentioned pixel array and a control circuit for controlling the pixel array to output at least one electrical stimulus to a retinal nerve cell on a single silicon substrate. In the embodiment, the control circuit includes a correlated double sampling (CDS) unit 20, an analog-to-digital converter (ADC) 30, a digital core 40, and a digital-to-analog converter (DAC) 50. Other suitable components may be further added to the control circuit as appropriate. In the embodiment, the individual operation of the components in the control circuit and the operation of the retinal implant device are similar to the embodiments described above except for being integrated in a single substrate. Thus, the detailed operation is not described herein.

What is claimed is:

1. A system for artificial retinal prosthesis, comprising:
a pixel array, comprising n sub-pixels converting incident light to electrical stimulation signals;
a correlated double sampling unit, having a communication connection with the pixel array electrically;
an analog-to-digital converter, coupled to the correlated double sampling unit and outputting a first digital signal;
a digital core, coupled to the analog-to-digital converter to receive the first digital signal and output a second digital signal after analysis; and
a digital-to-analog converter, coupled to the digital core to receive the second digital signal.

2. The system for artificial retinal prosthesis as claimed in claim 1, wherein each of the sub-pixels comprises a pixel electrode.

3. The system for artificial retinal prosthesis as claimed in claim 2, wherein after receiving the second digital signal, the digital-to-analog converter outputs an electrical stimulation signal related to light intensity to the pixel electrode to electrically stimulate at least one nerve cell.

4. The system for artificial retinal prosthesis as claimed in claim 2, wherein the pixel electrode is connected to the digital-to-analog converter and at least one nerve cell to transmit a signal sent by the digital-to-analog converter to the nerve cell to perform an electrical stimulus.

5. The system for artificial retinal prosthesis as claimed in claim 1, wherein n is a positive integer between 500 and 50,000.

6. The system for artificial retinal prosthesis as claimed in claim 1, wherein the pixel array, the correlated double sampling unit, the analog-to-digital converter, the digital core, and the digital-to-analog converter are integrated on a single substrate.

7. The system for artificial retinal prosthesis as claimed in claim 6, wherein the single substrate is a silicon substrate.

8. The system for artificial retinal prosthesis as claimed in claim 1, wherein the system for artificial retinal prosthesis is disposed in an epi-retina or a sub-retina of an eye structure.

9. The system for artificial retinal prosthesis as claimed in claim 1, wherein the system further comprises at least one decoder, the decoder is coupled to the pixel array to control reset, exposure and read out of the pixel array.

10. A system for artificial retinal prosthesis, comprising: a retinal implant device, the retinal implant device comprising a pixel array and a control circuit for controlling the pixel array to output at least one electrical stimulus to a retinal nerve cell, wherein the control circuit stimulates the retinal nerve cell row-to-row to reduce large transient currents.

11. The system for artificial retinal prosthesis as claimed in claim 10, wherein the pixel array comprises n sub-pixels for converting the incident light to the electrical stimulation signals, and n is a positive integer between 500 and 50,000.

12. The system for artificial retinal prosthesis as claimed in claim 10, wherein the pixel array and the control circuit are integrated on a single substrate.

13. The system for artificial retinal prosthesis as claimed in claim 12, wherein the single substrate is a silicon substrate.

14. The system for artificial retinal prosthesis as claimed in claim 12, wherein the system for artificial retinal prosthesis is disposed in an epi-retina or a sub-retina of an eye structure.

15. The system for artificial retinal prosthesis as claimed in claim 10, wherein the control circuit comprises a digital core that performs a row-to-row stimulation.
